(19) United States
(12) Patent Application Publication
(10) Pub. No.: US 2024/0189462 A1
(43) Pub. Date: Jun. 13, 2024

(54) FLUID DISPENSER STERILIZATION USING EXOGENIC ABSORBERS

(71) Applicant: TWENTY TWENTY THERAPEUTICS LLC, South San Francisco, CA (US)

(72) Inventor: Timothy Stowe, Alameda, CA (US)

(21) Appl. No.: 18/534,521

(22) Filed: Dec. 8, 2023

Related U.S. Application Data
(60) Provisional application No. 63/431,062, filed on Dec. 8, 2022.

(51) Int. Cl. A61L 2/00; A61F 9/00; A61K 9/08; A61K 41/17; A61L 2/26

(52) U.S. Cl. CPC A61L 2/0076; A61F 9/0008; A61K 9/08; A61K 41/17; A61L 2/0047; A61L 2/26; A61L 2202/11; A61L 2202/121; A61L 2202/122; A61L 2202/21

(57) ABSTRACT

A sterilizing fluid dispenser cartridge is described. The cartridge includes a reservoir configured to retain an aqueous fluid comprising an exogenic sterilizing light absorber; a dispensing head comprising a nozzle; and a polymeric nozzle cover movably coupled to the dispensing head. At least a region of the fluid dispenser cartridge is partially transparent. The fluid dispenser cartridge can be used as part of a fluid dispensing system, and methods of administering a sterilized aqueous fluid are also described.

### CROSS-REFERENCE TO RELATED APPLICATIONS

This application claims priority to U.S. Provisional Patent Application Ser. No. 63/431,062, filed Dec. 8, 2022, which is incorporated herein by reference.

### BACKGROUND

Ophthalmic formulations are required to pass very stringent sterility testing that show bacteria and fungi can't grow under long term storage conditions and several months of usage conditions. These requirements are imposed because infections caused by microorganisms in the eye can lead to substantial long-term consequences such as blindness.

In order to meet such requirements most over-the-counter (OTC) or prescription (Rx) ophthalmic formulations use broad spectrum preservatives to kill bacteria. Benzalkonium chloride (BAK+) is the most commonly used eye drop preservative. This preservative is in a class of organic salt molecules called quaternary ammonium compounds and is a cationic surfactant. Inevitably, preservatives such as BAK, though usually loaded into formulations typically at 50-150 ppm levels, still have low level toxicity to human cells which can lead to inflammatory immune responses causing dry eye disease.

In an attempt to overcome these issues, new bottles have been developed which have a one-way valve and a long fluid path channel to prevent the backflow of bacteria through the fluid path. These new bottles are referred to as Multi-Dose Preservative Free bottles or MDPF bottles and are sold by companies such as Nemera™ and Aptar™ Pharmaceuticals. These bottles all feature a one-way valve preventing backflow of liquid. In addition, these bottles also either feature a biofilter consisting of a 0.2 μm or smaller porous air permeable membrane for filtering air inlet to equalize pressure or a collapsible inner bottle bag so as to maintain pressure equality between the inside and outside of the bottle.

However, there are many challenges with such bottles. Conventional reusable eye drop systems are susceptible to bacterial and/or viral contamination especially at the outside of the tip which can come in direct contact with eyes and also with bathroom countertops. For example, contamination in and/or around a dispensing nozzle of a conventional eye drop system may travel with a dispensed drop into the patient's eye, causing infection. The outside tip can still be contaminated with bacteria as shown to be the case in 20% of the bottles tested, usually from accidental touching by patients with their hands or at the eye. Contamination with methicillin-resistant Staphylococcus aureus (MRSA) is a particularly serious problem and has been shown to be present in about 5% of bottles in some cases. Furthermore, this type of device does not lend itself well to be adapted to horizontal eye drop delivery methods.

TearClear™ Corp makes a filter which removes the preservatives like BAK before they enter the eye using a chemical filter which absorbs the preservative. However, this method requires higher squeezing forces, is difficult to use with higher viscosity formulations, and is not compatible with all drug formulations.

UV exposure can be used to kill and stop bacteria and fungi from growing. Maclean et al., J Blood Transfus. 2016:2016:2920514. This is usually the case because UV attacks cyclic amine groups in proteins in the cell and DNA which causes a breakdown in the cells reproductive machinery. However not all UV wavelengths are the same. Ultraviolet in the UVC (100-280 nm) and UVB (280-315 nm) are shorter in wavelength and can easily be absorbed in the walls of plastic bottles causing embrittlement without reaching bacteria inside the bottle. In addition, it has been shown that UVC and UVB can sometimes cause the loss of efficacy of ophthalmic Rx drugs such as latanoprost. UVA light (340-400 nm) can be just as effective as UVB and C when combined with exogenic absorbers. For example, riboflavin can be added to blood to provide in-situ UVA sterilization of the transfusion of donated blood to kill broad spectrums of viruses and bacteria. Hardwick et al., Photochem. Photobiol. 80 (3): 609-15 (2004).

In addition to UV light, deep blue wavelengths (400 nm-420 nm) have also been shown to kill bacteria. This wavelength mainly relies on porphyrin complex absorbers within the bacteria and requires continuous pulse trains or DC soaks in light. The mechanism works by creating reactive oxygen species (ROS) which can attack a bacteria's cellular machinery within its cell wall. Unfortunately, porphyrins may be limited in some bacteria and fungi and the mechanism is not as efficient as those that attack DNA at shorter wavelengths. Thus the current and energy required to continuously pulse or DC currents for deep blue LEDs can be prohibitively intensive, pre-maturely draining the energy stored in portable batteries.

What is needed is a new preservative-free method of eye dropper sterilization that is compatible with horizontal drop delivery, can be used with a wide spectrum of high and low viscosity eye drop formulations that suffers less from the limitations described herein.

### SUMMARY OF THE INVENTION

Most eyedroppers need eye irritating preservatives or expense complex preservative free packaging. The present invention uses a germicidal UV LED in the applicator and a low-cost cartridge for administering eye drops. The UV LED can also prevent growth of mold. In addition, the present invention includes exogenic absorbers of UVA light or deep blue light to improve the effectiveness of UV sterilization.

In one aspect, a fluid dispenser cartridge is provided. The fluid dispenser cartridge includes a chamber providing a reservoir configured to retain an aqueous fluid comprising an exogenic sterilizing light absorber; a dispensing head comprising a nozzle; and a polymeric nozzle cover movably coupled to the dispensing head, wherein at least a region of the fluid dispenser cartridge is partially transparent.

In another aspect, a fluid dispensing system is provided. The fluid dispensing system includes fluid dispenser cartridge comprising a chamber providing a reservoir configured to retain an aqueous fluid comprising an exogenic sterilizing light absorber; a dispensing head comprising a nozzle; and a polymeric nozzle cover movably coupled to the dispensing head, wherein at least a region of the fluid dispenser cartridge is partially transparent. The fluid dispensing system also includes an applicator configured to releasably retain the fluid dispensing cartridge, the applicator comprising a first opening configured to receive the fluid cartridge; and a second opening positioned adjacent the nozzle of the fluid cartridge when the fluid cartridge is disposed within the first opening; and a sterilizing light assembly positioned to illuminating the partially transparent region of the fluid dispenser cartridge with UVA light.

In another aspect, a method of administering a sterilized aqueous fluid is provided. The method includes exposing the nozzle of an aqueous fluid dispenser cartridge, wherein the aqueous fluid dispenser cartridge comprises a chamber providing a reservoir configured to retain an aqueous fluid comprising an exogenic sterilizing light absorber; a dispensing head comprising a nozzle; and a polymeric nozzle cover movably coupled to the dispensing head, wherein at least a region of the fluid dispenser cartridge is partially transparent; illuminating the partially transparent region of the fluid dispenser cartridge with UVA light; and administering a portion of aqueous fluid from the aqueous fluid dispenser cartridge to subject.

### BRIEF DESCRIPTION OF THE FIGURES

FIG. 1 is a perspective view of a fluid dispensing system in accordance with at least one embodiment of the present disclosure, the fluid dispensing system including a cartridge housed within an applicator.

FIG. 2 is a perspective view of the cartridge of FIG. 1, in accordance with at least one embodiment of the present disclosure.

FIG. 3 is a perspective cutaway view of the cartridge of FIG. 1, in accordance with at least one embodiment of the present disclosure.

FIG. 4 provides a graph showing the riboflavin absorption middle hump for UVA.

FIG. 5 provides a graph showing the UVA absorption hump in K series vitamins.

FIG. 6 is a perspective cutaway view of the cartridge and applicator of FIG. 1, including a sterilizing light assembly, in accordance with at least one embodiment of the present disclosure.

FIG. 7 is a diagrammatic illustration of the cartridge and applicator of FIG. 1, including a sterilizing light assembly, in accordance with at least one embodiment of the present disclosure.

### DETAILED DESCRIPTION OF THE INVENTION

The present invention provides a fluid containing an additive which is an exogenic optical absorbing ingredient that in-situ sterilizes the liquid formulation when UVA, Violet, or deep blue light is directed through the cartridge. The cartridge includes a chamber providing a reservoir configured to retain an aqueous fluid comprising a UVA light absorber; a dispensing head comprising a nozzle; and a polymeric nozzle cover movably coupled to the dispensing head. At least a region of the fluid dispenser cartridge is partially transparent to the light in this wavelength range. The fluid dispenser cartridge can be used as part of a fluid dispensing system, and methods of sterilizing the fluid dispenser cartridge are also provided.

The terminology as set forth herein is for description of the embodiments only and should not be construed as limiting of the invention as a whole. Unless otherwise specified, “a,” “an,” “the,” and “at least one” are used interchangeably. Furthermore, as used in the description of the invention and the appended claims, the singular forms “a”, “an”, and “the” are inclusive of their plural forms, unless contraindicated by the context surrounding such.

Also herein, the recitations of numerical ranges by endpoints include all numbers subsumed within that range (e.g., 1 to 5 includes 1, 1.5, 2, 2.75, 3, 3.80, 4, 5, etc.).

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

A “subject,” as used herein, can be any animal, and may also be referred to as the patient. Preferably the subject is a vertebrate animal, and more preferably the subject is a mammal, such as a research animal (e.g., a mouse or rat) or a domesticated farm animal (e.g., cow, horse, pig) or pet (e.g., dog, cat). In some embodiments, the subject is a human.

“Biocompatible” as used herein, refers to any material that does not cause injury or death to a subject or induce an adverse reaction in a subject when placed in contact with the subject's tissues. Adverse reactions include for example inflammation, infection, fibrotic tissue formation, cell death, or thrombosis. The terms “biocompatible” and “biocompatibility” when used herein are art-recognized and mean that the material is neither itself toxic to a subject, nor degrades (if it degrades) at a rate that produces byproducts at toxic concentrations, does not cause prolonged inflammation or irritation, or does not induce more than a basal immune reaction in the host.

As used herein, “treatment” means any manner in which the symptoms of a defect, condition, disorder, or disease, or any other indication, are ameliorated or otherwise beneficially altered.

“Sterilization,” as used herein, refers to decreasing or eliminating bacteria and/or other microorganisms from an object, such as a surface or an aqueous fluid. Sterilization includes eliminating at least 90%, at least 95%, or at least 99% of the bacteria and/or other microorganisms from an object.

### Sterilizing Fluid Dispenser Cartridge

In one aspect, the present invention provides a fluid dispenser cartridge. The fluid dispenser cartridge includes a chamber providing a reservoir configured to retain an aqueous fluid comprising a UVA light absorber; a dispensing head comprising a nozzle; and a polymeric nozzle cover movably coupled to the dispensing head, wherein at least a region of the fluid dispenser cartridge is partially transparent. The reservoir is preferably sterile and water-tight, and can have a volume ranging from about 2 mL to about 10 mL. UVA light generated by a surface mounted UVA LED can be directed through a transparent diaphragm or a nozzle cap of a fluid delivery cartridge.

In some embodiments, the fluid dispenser cartridge includes a single-use fluid chamber; i.e., the fluid dispensing cartridge is a replaceable cartridge that it is independent from the applicator/delivery device. The replaceable cartridge is coupled to a reusable dispensing head. In further embodiments, the device does not include a replaceable cartridge, but rather incorporate all the components of the replaceable cartridge into the applicator body. For example, in some embodiments, the nozzle and the polymeric nozzle cover are part of the applicator body, while the fluid reservoir is part of the removable cartridge. Embodiments including both the fluid chamber and the dispensing head together can be designed for single use.

In some embodiments, the polymeric nozzle cover comprises a thermoplastic elastomer that is at least partially transparent to UVA light having a wavelength of 340-415 nm, and UVA light is directed to the polymeric nozzle cover. However, in other embodiments, other regions of the fluid dispenser cartridge (e.g., the chamber) can be partially transparent, and UVA light directed to these other region(s). A material is partially transparent if at least a portion of light illuminating a surface is able to penetrate and shine through the material. The degree of transparency can be referred to by a percentage of light which is able to shine through the material. In some embodiments, the material is fully transparent (i.e., 100% transparent), while in other embodiments, the material is about 50% transparent, about 60% transparent, about 70% transparent, about 80% transparent, or about 90% transparent.

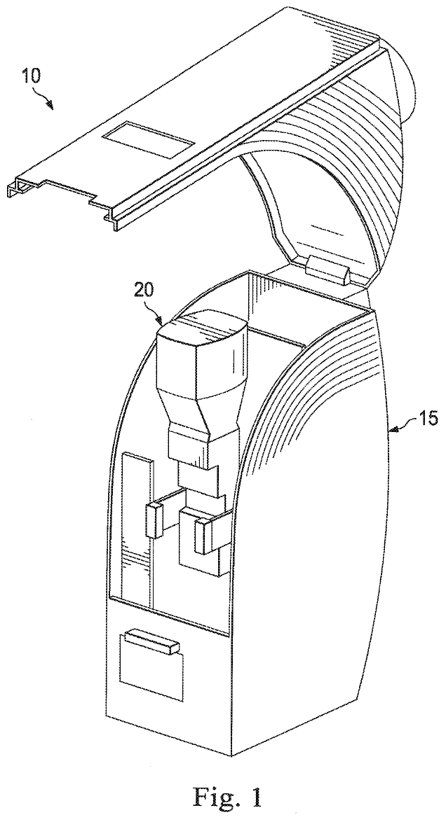

The fluid dispenser cartridge is typically included in an applicator, as shown in FIG. 1, which provides a perspective view of a fluid dispensing system 10 comprising a fluid dispenser cartridge 15 housed within an applicator 20.

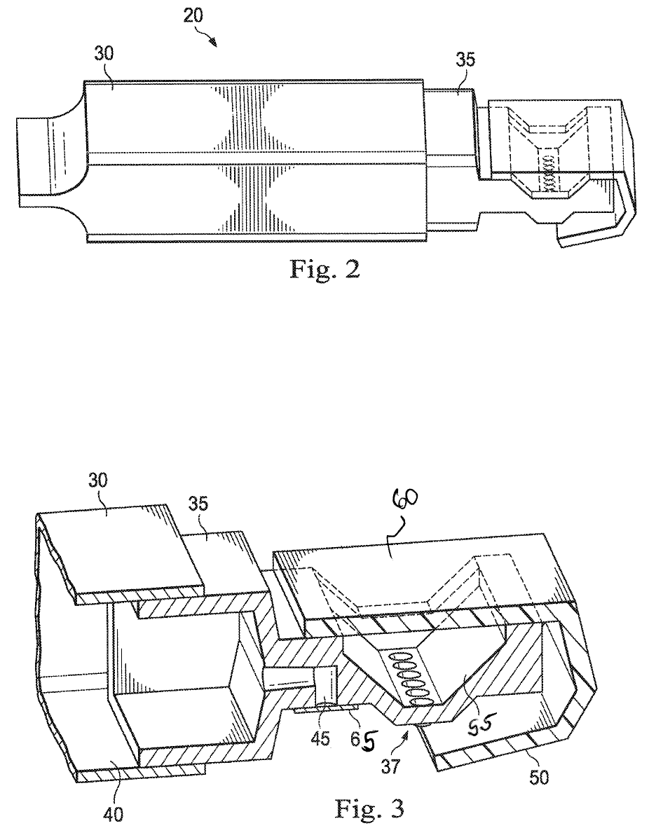

An example of a fluid dispenser cartridge suitable for use in the present invention is shown in FIG. 2, which provides a perspective view of a fluid dispenser cartridge. As illustrated, the fluid dispenser cartridge 20 includes a chamber 30 and a dispensing head 35 that is attached to the chamber 30. In some embodiments, the fluid dispenser cartridge 20 is about 14 mm wide×14 mm long×7 mm thick, however, the dimensions may vary.

Generally, and as illustrated in the cutaway view in FIG. 3, the chamber 30 encloses a a reservoir 40 in which an aqueous fluid comprising an exogenic sterilizing light absorber is accommodated. In some embodiments, the aqueous fluid is aseptically dispensed before the head 35 is heat sealed or coupled to the chamber 30.

As illustrated, the head 35 is coupled to the chamber 30 to dispense the aqueous fluid from the reservoir 40. Generally, the head 35 is at least temporarily in fluid communication with the reservoir 40 and forms a nozzle 37 and an air entry port 45. The head 35 also includes a polymeric nozzle cover 50 and a wall 60 that is movable relative to the nozzle 37. At least a region of the fluid dispenser cartridge 20 is partially transparent. The head 35 forms a holding chamber 55 that is in fluid communication with the reservoir 40 and that is positioned between the nozzle 37 and the nozzle cover 50. In some embodiments, the air entry port 45 is positioned between the nozzle 37 and the chamber 30. In some embodiments, the air entry port 45 is a sterile air filtered air entry port. A filter 65 may be positioned over the air entry port 45.

### UV Exposure and Exogenic Absorbers

UVA (315 nm-400 nm) wavelengths as well as violet and deep blue LEDs are special in that they do not directly attack most prescription drugs or biologics and can pass through the walls of ophthalmic bottles without causing embrittlement above about >340 nm in wavelength. Similar to the reactive oxygen species (ROS) mechanisms used by porphyrins, they are absorbed by natural vitamin complexes occurring in bacteria and fungi. Specifically, they absorb very well in riboflavin B2 or B2 derivatives such as FAD flavoprotein in Complex II, and vitamin K2, a naturally occurring substance in honey. Kvam, E. and Benner, K., J Photochem Photobiol B., 209, 111899 (2020). These mechanisms have been shown to be 10× more efficient than the porphyrin mechanism for deep violet or blue light. Mori et al., Med Biol Eng Comput., 45(12):1237-41 (2007).

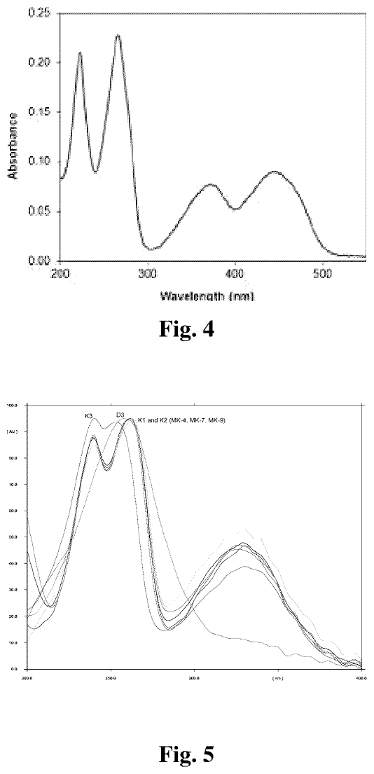

Exogenic absorbers of UVA light or deep blue in the (340 nm-415 nm range) such as Riboflavin (B2) or K2 menaquinones vitamins (vitamin K2 homologs commonly found in honey) are added to the ophthalmic eye drop formulation at levels of 10-1000 ppm by weight to provide an exogenic absorber in the range of 340-415 nm. A graph showing the riboflavin absorption middle hump for UVA is provided in FIG. 4, while a graph showing the UVA absorption hump in K series vitamins is provided in FIG. 5. In some embodiments, the UVA light absorber absorbs UVA light having a wavelength of 340-415 nm. In further embodiments, the UVA light absorber is riboflavin or a K2 vitamin menaquinone. In addition, the strong absorption spectrum of many naturally occurring vitamins such as riboflavin (B2) and K1 and especially K2 overlap the emission spectrum of many new high-efficiency UVA light emitting diodes (LEDs). Including an exogenic UVA light absorber allows for lower doses of UVA light to be used, which can increase batter life.

UVA light is very effective for killing a wide variety of microorganisms, as well as being safer for the user should UVA light leak from the UV eye dropper device. In some embodiments, the UVA light source is a light emitting diode (LED) surface mount source that supplies an optical intensity ranging from an optical intensity of 4 0 mW/cm² to about 100 mW/cm² in a pulsed or DC form. UVA light being in the range of 340-415 nm can easily be piped through clear Polyproylene, SEB (Styrene-ethylene-butylene-styrene TPEs) or SEP (Styrene-Ethylenc/Propylene Block Copolymer TPEs), polypropylene, or clear polyethylene or PET.

In some embodiments, a subrange of the UVA and/or deep blue light range (340 nm to 415 nm) can be used. For example, a subrange of UVA light corresponding to the main absorption region of the exogenic UVA absorber included in the aqueous fluid can be used. In some embodiments, light having a wavelength from about 340 nm to about 375 nm is used, while in further embodiments light having a wavelength from about 375 nm to 415 nm, or from 360 nm to 390 nm can be used.

The ocular safety and effectiveness of the UVA/Riboflavin vitamin mechanism has been shown for Photorefractive Intrastromal Cross-Linking surgery for the cornea with notably significant reductions in post-surgical infections owing to the natural sterilization processes of energized Riboflavin of the procedure.

The fluid dispenser cartridge includes a chamber providing a reservoir configured to retain an aqueous fluid. The aqueous fluid can be a wide variety of different water-based solutions. In some embodiments, the aqueous fluid is a biocompatible fluid, such as an aqueous fluid having a pH ranging from about 6 to about 8. In some the aqueous fluid is a pharmaceutical fluid. Pharmaceutical fluids may include an active agent, and can be used to treat a variety of conditions such as nasal conditions, pulmonary conditions, ocular conditions, and dermatological conditions. One of the advantages of the present invention is that the use of UV sterilization allows the aqueous fluid to be preservative-free. This is because once the UV light is turned off reactive oxygen species (ROS) quickly dissipate. The strength of the artificial UV light from the LED used in this method is much higher than the natural UVA sunlight experienced on a sunny day which has an intensity over the full integrated spectrum between 340-400 of roughly 3.6 mW/cm² or 10× lower than the light concentration seen inside the cartridge bottle from a UVA LED.

A pharmaceutical fluid is typically a pharmaceutically acceptable fluid. The phrase “pharmaceutically acceptable” refers to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art.

The fluids dispensed by the devices and systems described herein may be of various forms and properties. In some embodiments, the fluids dispensed by the devices and systems of the present disclosure include therapeutic and/or pharmaceutical fluids. For example, the fluid dispensing devices and systems may be configured to provide a stream of hyaluronic acid-based eye drops to the patient's eye to treat dry eye and/or other ophthalmic conditions. In other embodiments, the fluid dispensing devices and systems may be configured to provide saline solutions to the patient's eye. Glaucoma medications may also be used. The fluid dispensing devices and systems may be used to administer a variety of pharmaceutical and/or non-pharmaceutical agents to the patient. Further, the fluids dispensed by the devices and systems may have a variety of viscosities. In some embodiments, the fluid dispensing devices and systems described herein are configured to eject a stream of a gel-like substance to the patient's eye.

In some embodiments, the aqueous fluid is an ophthalmic solution (i.e., a sterile aqueous fluid formulated for administration to the eye). Examples of ophthalmic solutions include solutions used for liquid eye drops, and can be used to treat a variety of conditions such as eye infections, eye allergies, and corneal ulcers.

In some embodiments, the ophthalmic solution is formulated to provide lubrication to the eye (i.e., artificial tears). A lubricating ophthalmic solution contains an active ingredient which functions as a lubricating demulcent, preferably having a concentration around 0.1-2%, isotonic agents that add salts in the eye drop to balance the formulation with existing tear salts and balance osmotic forces, pH buffering agents to balance the pH with existing natural tears, cheating agents to get rid of any metal impurities. While prior art ophthalmic solutions typically include preservatives in the range of 0.001%-0.01% to inhibit any bacterial growth, the present formulations can be preservative free as a result of the UVA sterilization. In place of a preservative, the ophthalmic solutions used as an aqueous fluid for the present invention can include a low concentration (e.g., 0.1% or less) of an exogenic UVA absorbing compound such as riboflavin that does not cause irritation or toxicity in human cells.

### Sterilizing Fluid Dispensing System

Another aspect of the present invention provides a fluid dispensing system. The system comprises an applicator and a fluid dispenser cartridge. The fluid dispenser cartridge includes a chamber providing a reservoir configured to retain an aqueous fluid comprising: a standard pharmaceutical, OTC, or monograph formulation with an additional UVA light absorber; a dispensing head comprising a nozzle; and a polymeric nozzle cover movably coupled to the dispensing head, wherein at least a region of the fluid dispenser cartridge is partially transparent. In some embodiments, the UVA light absorber absorbs both UVA light as well as deep violet light having a wavelength range of 340-415 nm. In further embodiments, the polymeric nozzle cover comprises a thermoplastic elastomer that is at least partially transparent to UVA and deep violet light having a wavelength of 340-415 nm. The aqueous fluid can be composed of a variety of solutions, including pharmaceutical solutions. For example, in some embodiments, the aqueous fluid is an ophthalmic solution. Because the fluid dispensing system is sterilizing, in some embodiments the aqueous fluid formulation can be made without any preservatives such as BAK and can be thus categorized as preservative-free. In some embodiments, the fluid dispensing system can be hand-held.

The system also includes an applicator configured to releasably retain the fluid cartridge, the applicator comprising; a first opening configured to receive the fluid cartridge; and a second opening positioned adjacent the nozzle of the fluid cartridge when the fluid cartridge is disposed within the first opening; and a sterilizing light assembly positioned to illuminate the nozzle.

In some embodiments, the applicator further comprises a shutter coupled to the applicator and configured to articulate between a closed position and an open position, wherein the shutter is configured to actuate the polymeric nozzle cover to expose the nozzle when the shutter is articulated from the closed position to the open position, wherein the shutter comprises an inward-facing surface, wherein the inward-facing surface faces the nozzle when the shutter is in the closed position; and a sterilizing UVA light assembly coupled to the inward-facing surface of the shutter. The fluid dispenser system can supply the power to the sterilizing UVA LED such that it does not turn on unless the shutter door is closed.

In some embodiments, the sterilizing UVA light assembly comprises a sterilizing light element and a pair of electrical conductors electrically coupled to the sterilizing light element, wherein the pair of electrical conductors are configured to contact with the pair of electrical contacts when the shutter assembly is in the closed position. In further embodiments, the system includes a power supply coupled to the applicator and configured to provide electrical power to the sterilizing light assembly via the pair of electrical contacts. In yet further embodiments, the system includes a controller configured to cause the power supply to provide the electrical power to the sterilizing light assembly for one or more periods of time.

In some embodiments, the fluid dispensing system may include more than one sterilizing component. For example, the fluid dispensing device may include an array of 2 or more micro LEDs. In some embodiments, the LEDs may include focusing optics configured to focus and/or direct UV light to a desired area on the dispensing head. In some embodiments, the fluid dispensing device may include one or more sensors to detect whether the fluid cartridge is loaded into the dispensing device. For example, the fluid dispensing device may be configured to activate the sterilizing components on the condition that the fluid cartridge is loaded into the system, as indicated by the sensors. The fluid cartridge detecting sensor may include an optical sensor, mechanical sensor, magnetic, capacitive and/or any other suitable type of sensor.

The sterilization components are positioned, oriented, and configured to emit sterilizing radiation toward and through the partially transparent region of the fluid dispensing cartridge. In some aspects, the nozzle cover includes a plurality of oblique surfaces oriented at nonparallel and non-perpendicular angles relative to the axes of the sterilization components. The oblique surfaces of the nozzle cover may be configured to refract at least a portion of the sterilizing radiation (e.g., UV light) toward the nozzle to sterilize the nozzle and the area around the nozzle. Accordingly, the oblique refracting surfaces of the nozzle cover may advantageously provide a wide scattering area of the sterilizing radiation in and around the nozzle.

The sterilizing light assembly may include a light element configured to sterilize the nozzle and/or one or more components adjacent to the nozzle, such as the nozzle cover, and/or the fluid retained within the dispensing head. For example, the light assembly may comprise a UVA LED, a violet LED, and/or any other suitable light source for sterilizing the nozzle and/or aqueous fluid. The light guide may comprise a surface reflective in at least one of the UV spectrum or the violet spectrum such that sterilizing light rays from the light assembly are directed along the dispensing axis and toward a back side of the nozzle. In some aspects, the light assembly may comprise more than one light element. For example, the light assembly may comprise an aiming light source for positioning and orienting the dispensing device, and a sterilizing light source for sterilizing the nozzle, the nozzle cover, and/or the aqueous fluid. The sterilizing components may include other types of light sources, including incandescent bulbs, halogen bulbs, and/or any other suitable type of light source.

In some embodiments, the sterilizing light assembly may be configured to emit UVA-I and/or violet light. For example, a sterilization component (e.g., LED) may be configured to emit light having wavelengths of 340 nm-415 nm. Accordingly, the sterilizing UVA light assembly may be configured to emit UVA light, visible light, or both. In one example, the sterilization component may include a surface mount (SMD) UVA LED at 385 nm with up to 100 mA from Würth Elektronik part #153283387A212 or an SMDt Everlight SMT ELUA35350G5-P6070U13240500-VDIM at 365 nm with up to 500 mA of current.

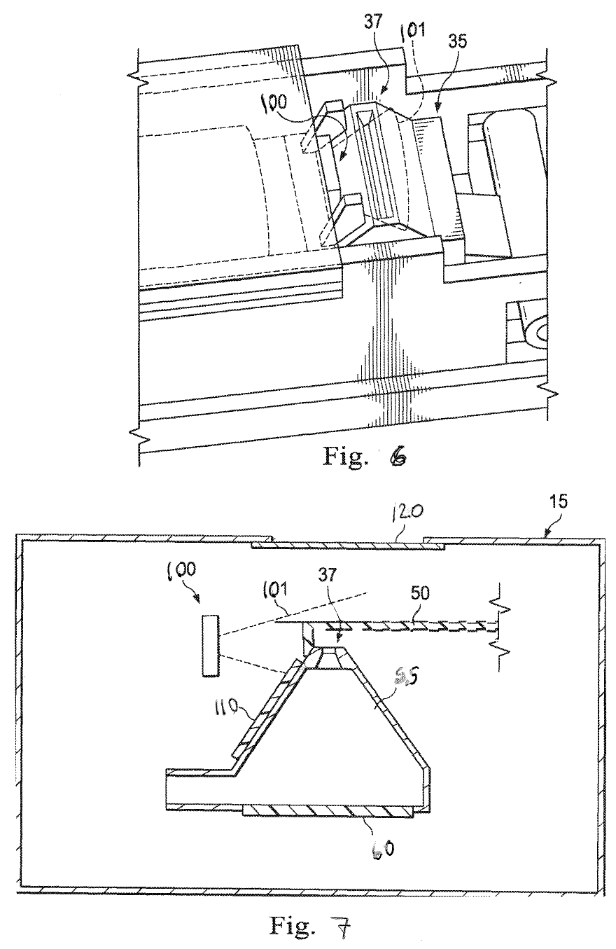

As illustrated in FIGS. 6 and 7, a sterilizing light assembly 100 is included that can consist of one or two ultraviolet (“UV”) light emitting diodes (“LEDs”) that are positioned relative to the nozzle 37 such that the nozzle 37 is exposed to an LED light cone 101 through either the tip of the nozzle head or the cap 50. In addition, and in some embodiments, because constant power to the UV LED uses a substantial amount of battery energy, the UV LED can be turned on just after an application to the eye. Due to the close proximity of the UV LED to the nozzle 37, only a few seconds of exposure is necessary for sterility using the appropriate wavelength. At the appropriate UVC wavelength range of 285 nm for example, the UV LED is known to kill viruses, bacteria, and even molds very effectively with over 103 reductions with only millijoules of energy over a concentrated close proximity area. In the embodiments in which the cap 50 extends between the nozzle 37 and the sterilizing light source 100, for example as illustrated in FIG. 7, the cap 50 is at least partially transparent to the UV wavelength and is made from UV stabilized materials. Note that while FIGS. 6 & 7 show a sterilizing light assembly positioned to illuminate the nozzle, that the sterilizing light assembly can also be positioned to illuminate the aqueous fluid through a region of the chamber that is at least partially transparent.

In some embodiments, a UV shield 110 is applied over a portion of the nozzle 37 or other portion of the head 35. For example, the UV shield 110 may include a thin layer of sputtered SiO₂ or metal to prevent a portion of the nozzle 37 from exposure to the UV light. In some embodiments, the UV shield 370 prevents the potential for degradation of a drug component of the viscous fluid in the main holding chamber 55 and only affects a small concentrated area around the nozzle.

As illustrated in FIG. 6, an example sterilizing light assembly 100 that includes a SMD UV LED is coupled to the applicator 15 and situated near the nozzle 37. An example of a SMD UV LED includes for example the L944-UV265-4 265 nm domed UVC LED from American Opto Plus LED. In addition to UV-C LEDs killing bacteria, UV-C LEDs also kill mold spores. While described as a sterilizing light assembly 100, the sterilizer 300 is not required to kill all of the bacteria, viruses, and fungi. Instead, the sterilizing light assembly 100 may kill or reduce a significant portion of the bacteria, viruses, and fungi by several orders of magnitude. In some cases, this may result in ocular drug formulations which can be completely preservative free, which is highly desirable. In other cases, the use of preservatives may be dramatically reduced. It should be noted that under operations, the nozzle cover 50 is not touched or removed by the user from the cartridge in any way but is permanently tethered to it at some point, and is held back further from the eyelashes than a normal eye dropper. A dust cover 120 of the applicator 15 also keeps the nozzle cover or cap 50 clean and prevents UV from leaking outside the applicator when the sterilizer 300 is turned on. The only chance for biological contamination is an airborne event during liquid dispensing. However, the diffusion time and diffusion rates for even the fastest self-propelled bacteria is slow enough so as to be killed by the UV light near the nozzle 37 before any growth can take place. Additionally, the nozzle 37 is covered internally by the elastomeric wall 60 after dispensing events, which acts as a valve trapping any such biological contamination. In some embodiments, the wall 60 can remain against the nozzle 37 until after a brief UV exposure has taken place.

In some embodiments, the fluid dispensing system may further include an actuator assembly comprising an applicator configured to releasably retain the fluid cartridge. The applicator may include: a first opening configured to receive the fluid cartridge; and a second opening positioned adjacent the nozzle of the fluid cartridge when the fluid cartridge is disposed within the first opening. The fluid dispensing system may further include a shutter coupled to the applicator and configured to articulate between a closed position and an open position. In some embodiments, the shutter is configured to actuate the polymeric nozzle cover to expose the nozzle when the shutter is articulated from the closed position to the open position. In some embodiments, the shutter comprises an inward-facing surface, wherein the inward-facing surface faces the nozzle when the shutter is in the closed position.

In some embodiments, the dispensing head further includes a compression membrane on a first side of the dispensing head, and wherein the drop actuator assembly further comprises a solenoid configured to strike the compression membrane to expel fluid through the nozzle. In some aspects it may be advantageous for the solenoid to be of a bi-stable or two position latching type. A bi-stable solenoid or a latching-type solenoid may maintain both the latched and unlatched positions without constant current draw so that current is supplied only during the transition from one state to another. In addition, the force and speed and momentum of the movement with which the plunger strikes may be adjusted electronically in solenoid designs by varying the amount of current supplied. Thus, higher de-latching current may provide a higher strike force for higher viscosity liquids formulations inside the cartridge to maintain a dosage for a variety of fluid viscosities. It may be advantageous for the solenoid to reduce or eliminate rebound so as not to resonate back and forth or bounce as this can draw outside air inside the nozzle opening during immediate retraction or bounce thus preventing or hindering the potential use of preservative-free formulations. Thus a solenoid with a damping feature such as a soft elastic dampener to eliminate rebound motion may be used. Combining the dampener with a bistable operation may allow for the outward push state to be maintained at the end of travel without any immediate retractions when current is turned off. The compression membrane can act as an internal elastic nozzle cover as well as dampener on the inside of the nozzle blocking penetration of external pathogens. For eliminating such unwanted ingress, the external nozzle cover may be changed to the closed position before the internal compression membrane is pulled away to return to its open or retracted position by moving the solenoid from an outward pushed state to a retracted pulled state. Thus a bistable solenoid with adjustable strike force having an end point dampener may be an advantageous configuration for a micro stream ejecting actuator.

To coordinate the opening and closing of the nozzle cap with the shutter opening in the applicator it may be advantageous to include a coupling feature between these two elements. In some embodiments, the fluid cartridge further comprises an arm coupled to the polymeric cover, where the arm is movable between an open state and a closed state, and where the nozzle is exposed when the arm is in the open state. In some embodiments, the shutter includes a nozzle cover disengagement feature configured to actuate the arm to the open state. In some embodiments, the arm is spring-biased such that the polymeric cover is in the closed state when the shutter is in the closed position. The spring can provide extra sealing pressure on the external nozzle cap when disengaged for a tighter seal. In some embodiments, the fluid dispensing system may further include a mechanical actuator coupled to the shutter, where the mechanical actuator is configured to actuate the shutter to move from the closed position to the open position. In some embodiments, the mechanical actuator is configured to actuate the polymeric cover to move from the closed state to the open state.

To eliminate and lower the ingress of any outside bacterial pathogens for preservative free formulations, the fluid dispensing devices and systems described herein may be configured with brief times of exposure to the external environment during ejection of micro stream drops of material. In some aspects, pathogens—even those with self-locomotion mechanisms like flagella—may have limited mobility and may not travel significant distances upstream in a short time period. Accordingly, in this time period it may be possible to sterilize these pathogens provided sufficient sterilization dosage can be provided.

In some embodiments, the fluid dispensing system further includes: a pair of conductors coupled to the shutter and the sterilizing light assembly; a pair of electrical contacts coupled to the applicator, where the pair of electrical contacts are positioned to be in contact with the pair of conductors when the shutter is in the closed position; and a power supply coupled to the applicator and configured to provide electrical power to the sterilizing light assembly via the pair of electrical contacts. In some embodiments, the fluid dispensing system further includes a controller configured to cause the power supply to provide the electrical power to the sterilizing light assembly for one or more periods of time.

According to another embodiment of the present disclosure, a fluid dispensing device includes an applicator defining an opening, where the applicator comprises a pair of electrical contacts adjacent to the opening. The fluid dispensing device may further include a shutter assembly movably coupled to the applicator, where the shutter assembly includes a first shutter component; and a mechanical motorized or hand-triggered actuator coupled to the applicator and the first shutter component. In some embodiments, the mechanical actuator is movable from a first position to a second position, where the mechanical actuator is configured to cause the shutter assembly to move from an open position to a closed position when the mechanical actuator moves from the first position to the second position. In some embodiments, the fluid dispensing device further includes a sterilizing subassembly coupled to the first shutter component, where the sterilizing subassembly comprises: a sterilizing light element; and a pair of electrical conductors electrically coupled to the sterilizing light element. In some aspects, the pair of electrical conductors are configured to contact with the pair of electrical contacts when the shutter assembly is in the closed position.

In some embodiments, the mechanical actuator is coupled to the first shutter component via a second shutter component, wherein the first shutter component is rotatably coupled to the second shutter component by a hinge. In some embodiments, the first shutter component is positioned to obscure the opening when the shutter assembly is in the closed position. In some embodiments, the applicator comprises a track feature, and the first shutter component comprises a guide feature positioned at least partially within the track feature. In some embodiments, the guide feature is configured to slide within the track feature when the shutter assembly moves from the open position to the closed position.

### Sterilization Method

Another aspect of the present invention provides a method of administering a sterilized aqueous fluid to a subject. The method includes exposing the nozzle of an aqueous fluid dispenser cartridge, wherein the aqueous fluid dispenser cartridge, comprises a reservoir configured to retain an aqueous fluid comprising a exogenic light absorber; a dispensing head comprising a nozzle; and a polymeric nozzle cover movably coupled to the dispensing head, wherein at least a region of the fluid dispenser cartridge is partially transparent; illuminating the partially transparent region of the fluid dispenser cartridge with sterilizing light; and administering a portion of aqueous fluid from the aqueous fluid dispenser cartridge to subject. The method of sterilizing an aqueous fluid dispenser cartridge can include sterilizing the aqueous fluid within the reservoir, the portion of aqueous fluid being administered, and/or at least a region of the aqueous fluid dispenser cartridge (e.g., the nozzle region).

In some embodiments, the partially transparent region of the fluid dispenser cartridge is the polymeric nozzle cover. When the partially transparent region of the fluid dispenser cartridge is the polymeric nozzle cover, at least this region of the fluid dispenser cartridge should be illuminated with sterilizing light. In some embodiments, the sterilizing light has a wavelength UVA of 340-415 nm.

In some embodiments, the step of illuminating with sterilizing light comprises administering a pulse from an LED light source. For example, a controller and/or power supply of the dispensing device may be configured to control the sterilization component to emit light with a pulsing pattern. The pulsing pattern may increase the efficiency and reduce power consumption. In some embodiments, a sterilization component may be pulsed with a pulse frequency ranging between 50 Hz-2000 Hz. In one embodiment, the sterilization component may be pulsed with a pulse frequency ranging between 100 Hz-1000 Hz. In some aspects, a pulse frequency of less than 1000 Hz, for example, may allow the sterilization component to be pulsed with a higher optical intensity and without creating excess heat. In some aspects, pulsing between 25%-40% duty cycle may advantageously increase the efficacy and/or efficiency of the sterilization protocol.

Using a pulsed pattern as opposed to a continuous wave pattern may increase the effectiveness of the sterilization procedure. In this regard, some biological contaminants such as bacteria may include porphyrins which absorb visible light. In some aspects, the wavelengths of light mentioned above may target intracellular porphyrin molecules. The porphyrins may absorb the light and create free radicals that can attack both gram positive and negative bacterial walls. Accordingly, pulsing the light source may allow the porphyrins to better absorb light by giving them time to relax from an energized chemical state to a ground state, and in particular more efficiently create free radicals causing the bacterial walls to decay. It will be understood that pulsing may be used with visible or non-visible sterilizing light sources within the scope of the present disclosure.

The step of illuminating the partially transparent region of the fluid dispenser cartridge with sterilizing LED light can be done at a variety of different times. Dosing can occur after a shutter opening event or periodically throughout the day. Dosing with UVA need not be continuous. Pulsing may be more energy efficient. This gives complexes time to fall from an energized state thus releasing a reactive oxygen species that is responsible for the kill mechanism.

In some embodiments, illumination is carried out proximal in time to administering a portion of aqueous fluid from the aqueous fluid dispenser cartridge to subject. For example, in some embodiments, illumination is done immediately before and/or after administering a portion of the aqueous fluid.

In some embodiments, the illumination is used in a programmed manner. “Programmed” as used herein indicates that illumination can be turned on or off as a result of the device being programmed in firmware to provide illumination just after an eye drop dispensing event or before an eye drop dispensing event. Because there is not likely to be bacterial ingress at other times, there may be no need to do sterilizing exposure at other times, thus saving on battery life.

In other embodiments, illumination can be continuous, or periodic. For example, if a person is taking an eye drop with antibiotics, due to an eye infection, it may be advantageous to leave the sterilizing light in the applicator on for longer periods in order to have a secondary mechanism to kill bacteria and eliminate the possibility of bacteria growing resistant to the antibiotic in solution.

The aqueous fluid can be administered to one or more different locations on the subject, depending on the nature of treatment being provided. For example, aqueous fluids can be administered to the eyes, nose, lungs, mouth, ears, rectum, vagina, or skin of a subject. In some embodiments, the aqueous fluid is an ophthalmic solution, and the ophthalmic solution is administered to the eye (or eyes) of the subject.

Embodiments and aspects of the present disclosure, including the fluid dispensing devices and fluid cartridges, may include one or more features of the systems, devices, and/or methods described in U.S. Pat. No. 11,723,797, and U.S. Patent Publication No. 2021/0353458, the disclosures of which are incorporated herein by reference. For example, in some embodiments the fluid dispensing device is adapted to provide improved performance with high viscosity ophthalmic formulations, while in other embodiments the fluid dispensing device provides light-assisted alignment and aiming.

The complete disclosure of all patents, patent applications, and publications, and electronically available materials cited herein are incorporated by reference. Any disagreement between material incorporated by reference and the specification is resolved in favor of the specification. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A fluid dispenser cartridge, comprising:
a chamber providing a reservoir configured to retain an aqueous fluid comprising an exogenic sterilizing light absorber;
a dispensing head comprising a nozzle; and
a polymeric nozzle cover movably coupled to the dispensing head,
wherein at least a region of the fluid dispenser cartridge is partially transparent.

2. The fluid dispenser cartridge of claim 1, wherein the exogenic sterilizing light absorber absorbs UVA light having a wavelength of 340-440 nm.

3. The fluid dispenser cartridge of claim 1, wherein the exogenic sterilizing light absorber is riboflavin or a K2 vitamin menaquinone.

4. The fluid dispenser cartridge of claim 1, wherein the polymeric nozzle cover comprises a thermoplastic elastomer that is at least partially transparent to sterilizing light having a wavelength of 340-440 nm.

5. The fluid dispenser cartridge of claim 1, wherein the aqueous fluid is an ophthalmic solution.

6. The fluid dispenser cartridge of claim 1, wherein the aqueous fluid is preservative-free.

7. A fluid dispensing system, comprising:
A fluid dispenser cartridge comprising:
a chamber providing a reservoir configured to retain an aqueous fluid comprising:
an exogenic sterilizing light absorber;
a dispensing head comprising a nozzle; and
a polymeric nozzle cover movably coupled to the dispensing head,
wherein at least a region of the fluid dispenser cartridge is partially transparent;
an applicator configured to releasably retain the fluid dispenser cartridge, the applicator comprising:
a first opening configured to receive the fluid cartridge; and
a second opening positioned adjacent the nozzle of the fluid cartridge when the fluid cartridge is disposed within the first opening; and
a sterilizing light assembly positioned to illuminate the partially transparent region of the fluid dispenser cartridge with UVA light.

8. The fluid dispensing system of claim 7, further comprising a shutter coupled to the applicator and configured to articulate between a closed position and an open position, wherein the shutter is configured to actuate the polymeric nozzle cover to expose the nozzle when the shutter is articulated from the closed position to the open position, wherein the shutter comprises an inward-facing surface, wherein the inward-facing surface faces the nozzle when the shutter is in the closed position, and wherein the sterilizing light assembly coupled to the inward-facing surface of the shutter.

9. The fluid dispensing system of claim 7, wherein the exogenic sterilizing light absorber absorbs light having a wavelength of 340-440 nm.

10. The fluid dispensing system of claim 7, wherein the polymeric nozzle cover comprises a thermoplastic elastomer that is at least partially transparent to UVA light having a wavelength of 340-440 nm.

11. The fluid dispensing system of claim 7, wherein the aqueous fluid is an ophthalmic solution.

12. The fluid dispensing system of claim 7, wherein the aqueous fluid is preservative-free.

13. The fluid dispensing system of claim 7, wherein the sterilizing light assembly comprises:
a sterilizing light element; and
a pair of electrical conductors electrically coupled to the sterilizing light element, wherein the pair of electrical conductors are configured to contact with the pair of electrical contacts when the shutter assembly is in the closed position.

14. The fluid dispensing system of claim 13, further comprising a power supply coupled to the applicator and configured to provide electrical power to the sterilizing light assembly via the pair of electrical contacts.

15. The fluid dispensing system of claim 14, further comprising a controller configured to cause the power supply to provide the electrical power to the sterilizing light assembly for one or more periods of time.

16. A method of administering a sterilized aqueous fluid is provided, comprising:
exposing the nozzle of an aqueous fluid dispenser cartridge, wherein the aqueous fluid dispenser cartridge comprises a chamber providing a reservoir configured to retain an aqueous fluid comprising an exogenic sterilizing light absorber; a dispensing head comprising a nozzle; and a polymeric nozzle cover movably coupled to the dispensing head,
wherein at least a region of the fluid dispenser cartridge is partially transparent;
illuminating the partially transparent region of the fluid dispenser cartridge with UVA light; and
administering a portion of aqueous fluid from the aqueous fluid dispenser cartridge to subject.

17. The method of claim 16, wherein the transparent region of the fluid dispenser cartridge is the polymeric nozzle cover.

18. The method of claim 16, wherein the subject is a human subject.

19. The method of claim 16, wherein the UVA light has a wavelength of 340-440 nm.

20. The method of claim 16, where the step of illuminating with UVA light comprises administering a pulsed light.

21. The method of claim 16, wherein the aqueous fluid is an ophthalmic solution, and the ophthalmic solution is administered to the eye of the subject.

22. The method of claim 16, wherein illuminating the partially transparent region of the fluid dispenser cartridge with UVA light is done immediately before and/or after administering a portion of the aqueous fluid.
